(19) United States
(12) Patent Application Publication
(10) Pub. No.: US 2006/0019259 A1
(43) Pub. Date: Jan. 26, 2006

(54) Characterization of biopolymers by resonance tunneling and fluorescence quenching

(76) Inventor: Timothy H. Joyce, Mountain View, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/898,586

(22) Filed: Jul. 22, 2004

(51) Int. Cl. C12Q 1/68; C12M 3/04

(52) U.S. Cl. 435/6; 435/287.2

(57) ABSTRACT

The present invention provides a method and apparatus for determining the identity of a monomeric residue of a biopolymer. The apparatus comprises a substrate having a nanopore, a potential-producing element for producing a ramped potential across electrodes adjacent to the nanopore, and a quenchable excitable moiety adjacent to the nanopore. As a biopolymer passes through the nanopore, the identity of monomeric residues of a biopolymer may be determined by detecting changes in (a) current across the electrodes and (b) a signal of the quenchable excitable molecule. The subject method and apparatus find use in determining the identity of a plurality of monomeric residues of a biopolymer, and, as such, may be employed in a variety of diagnostic and research applications.

### BACKGROUND

Techniques for manipulating matter at the nanometer scale (“nanoscale”) are important for many electronic, chemical and biological purposes (See Li et al., “Ion beam sculpting at nanometer length scales”, Nature, 412: 166-169, 2001). Among such purposes are the desire to more quickly sequence biopolymers such as DNA. Nanopores, both naturally occurring and artificially fabricated, have recently attracted the interest of molecular biologists and biochemists for the purpose of DNA sequencing.

It has been demonstrated that a voltage gradient can drive a biopolymer such as single-stranded DNA (ssDNA) in an aqueous ionic solution through a naturally occurring transsubstrate channel, or “nanopore,” such as an α-hemolysin pore in a lipid bilayer. (See Kasianowicz et al., “Characterization of individual polynucleotide molecules using a membrane channel”, Proc. Natl. Acad. Sci. USA, 93: 13770-13773, 1996). The process in which the DNA molecule goes through the pore has been dubbed “translocation”. During the translocation process, the extended biopolymer molecule blocks a substantial portion of the otherwise open nanopore channel. This blockage decreases the ionic electrical current flow occurring through the nanopore in the ionic solution. The passage of a single biopolymer molecule can, therefore, be monitored by recording the translocation duration and the decrease in current. Many such events occurring sequentially through a single nanopore provide data that can be plotted to yield useful information concerning the structure of the biopolymer molecule. For example, given uniformly controlled translocation conditions, the length of the individual biopolymer can be estimated from the translocation time.

One desire of scientists is that the individual monomers of the biopolymer strand might be identified via the characteristics of the blockage current, but this hope may be unrealized because of first-principle signal-to-noise limitations and because the naturally occurring nanopore is thick enough that several monomers of the biopolymer are present in the nanopore simultaneously.

More recent research has focused on fabricating artificial nanopores. Ion beam sculpting using a diffuse beam of low-energy argon ions has been used to fabricate nanopores in thin insulating substrates of materials such as silicon nitride (See Li et al., “Ion beam sculpting at nanometer length scales”, Nature, 412: 166-169, 2001). Double-stranded DNA (dsDNA) has been passed through these artificial nanopores in a manner similar to that used to pass ssDNA through naturally occurring nanopores. Current blockage data obtained with dsDNA is reminiscent of ionic current blockages observed when ssDNA is translocated through the channel formed by α-hemolysin in a lipid bilayer. The duration of these blockages has been on the millisecond scale and current reductions have been to 88% of the open-pore value. This is commensurate with translocation of a rod-like molecule whose cross-sectional area is 3-4 nm² (See Li et al., “Ion beam sculpting at nanometer length scales”, Nature, 412: 166-169, 2001). However, as is the case with single-stranded biopolymers passing through naturally occurring nanopores, first-principle signal-to-noise considerations make it difficult or impossible to obtain information on the individual monomers in the biopolymer.

A second approach has been suggested for detecting a biopolymer translocating a nanopore in a rigid substrate material such as Si₃N₄. This approach entails placing two tunneling electrodes at the periphery of one end of the nanopore and monitoring tunneling current from one electrode, across the biopolymer, to the other electrode. However, it is well known that the tunneling current has an exponential dependence upon the height and width of the quantum mechanical potential barrier to the tunneling process. This dependence implies an extreme sensitivity to the precise location in the nanopore of the translocating molecule. Both steric attributes and physical proximity to the tunneling electrode could cause changes in the magnitude of the tunneling current which would be far in excess of the innate differences expected between different monomers under ideal conditions. For this reason, it is difficult to expect this simple tunneling configuration to provide the specificity required to perform biopolymer sequencing.

Resonant tunneling effects have been employed in various semiconductor devices including diodes and transistors. For instance, U.S. Pat. No. 5,504,347, Javanovic, et al., discloses a lateral tunneling diode having gated electrodes aligned with a tunneling barrier. The band structures for a resonant tunneling diode are described wherein a quantum dot is situated between two conductors, with symmetrical quantum barriers on either side of the quantum dot. The resonant tunneling diode may be biased at a voltage level whereby an energy level in the quantum dot matches the conduction band energy in one of the conductors. In this situation the tunneling current between the two conductors versus applied voltage is at a local maximum. At some other bias voltage level, no energy level in the quantum dot matches the conduction band energy in either of the conductors and the current versus applied voltage is at a local minimum. The resonant tunneling diode structure can thus be used to sense the band structure of energy levels within the quantum dot via the method of applying different voltage biases and sensing the resulting current levels at each of the different voltage biases. The different applied voltage biases can form a continuous sweep of voltage levels, and the sensed resulting current levels can form a continuous sweep of current levels. The slope of the current versus voltage can in some cases be negative. Conceptually, it is also possible to inject a known current between the conductors and measure the resulting voltage, but this approach can fail if the characteristic current versus voltage has a negative slope region. For this reason, applying a known voltage bias and sensing the resultant current is usually the preferred method.

The problem with many of these techniques regards the ability to actually obtain measurements from the biopolymers that translocate through nanopores. Theoretically, these systems should be capable of detecting and recording information that can distinguish one monomer from another. However, to date no concrete experimental data exists to show that this is actually possible. Therefore, there is a need for alternate systems and methods for identifying, detecting and characterizing biopolmers. In addition, there is a need for a system or method that may record and capture information traversing nanopores on a time scale of less than a microsecond. A number of techniques and systems have been employed for probing molecules on rapid time scales using fluorescence, phosphorescence or bioluminescense. These techniques often employ the use of a fluorophore or chromophore in a protein and a quencher molecule. A number of quencher molecules have been identified for probing protein and nucleic acids structures. For instance, some known quenchers include coumarin, fluorescein, cesium chloride, potassium iodide, oxygen, and quinaldic acid. Chromophores in proteins include aromatic amino acids such as tryptophan, phenylalanine, tyrosine and histidine. In nucleic acids, a number of studies have been conducted using guanine as a fluorophore.

The problem with many phosphorescence or fluorescence techniques is that they become rather difficult to control how and when a quencher molecule contacts a fluorophore or chromophore. In addition, for collisional quenching to take place the actual molecules need to contact or come within close proximity. In some systems that use chromophores, the excited molecules have been shown to transfer energy from the excited molecule to another molecule close by or in the vicinity. For instance, studies have been conducted using metals such as lanthanum or terbium to bind to calcium binding loops of proteins (EF hand calcium binding loop). The chromophore can then be excited and energy can be transferred to the metals from the chromophore by an energy transfer process. Both Dexter and Forster energy transfer models describe these energy transfer processes for different fluorophore to quencher distances. Energy transfer is contingent upon the proximity of the metal to the chromophore in the molecule. A resultant energy is emitted from the metals at defined wavelengths that are characteristic of the structure of the biomolecule. In other words, both excitation and emission spectra can be developed that show varying line shapes that are characteristic of a particular biomolecule.

The references cited in this application infra and supra, are hereby incorporated in this application by reference. However, cited references or art are not admitted to be prior art to this application.

### SUMMARY OF THE INVENTION

The present invention provides a method and apparatus for determining the identity of a monomeric residue of a biopolymer. The apparatus comprises a substrate having a nanopore, a potential-producing element for producing a ramped potential across electrodes adjacent to the nanopore, and a quenchable excitable moiety adjacent to the nanopore. As a biopolymer passes through the nanopore, the identity of monomeric residues of a biopolymer may be determined by detecting changes in (a) current across the electrodes and (b) a signal of the quenchable excitable molecule. The subject method and apparatus find use in determining the identity of a plurality of monomeric residues of a biopolymer, and, as such, may be employed in a variety of diagnostic and research applications.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 illustrates theoretical results obtained from the first signal producing system of a subject apparatus. The voltage at which a monomeric residue causes resonance tunneling (i.e., an increase in current) indicates the identity of monomeric residues.

FIG. 2 illustrates theoretical results obtained from the second signal producing system of a subject apparatus. The amplitude of a signal obtained from the quenchable excitable moiety changes as different monomeric residues of a biopolymer pass through the nanopore as a resulting of quenching.

FIG. 3 schematically illustrates a first embodiment of the present invention.

FIG. 4A schematically illustrates a second embodiment of the present invention.

FIG. 4B schematically illustrates a third embodiment of the present invention.

FIG. 5A schematically illustrates a fourth embodiment of the present invention.

FIG. 5B schematically illustrates a fifth embodiment of the present invention.

FIG. 6A schematically illustrates a sixth embodiment of the present invention.

FIG. 6B schematically illustrates a sixth embodiment of the present invention.

FIG. 7 schematically illustrates a one dimensional quantum mechanical potential model of a physical electrode nanopore system.

FIG. 8 schematically illustrates resonant tunneling conditions for a one-dimensional double-barrier quantum mechanical model.

FIG. 9 shown a representative plot of an expected resonant tunneling current spectrum as a function of time (alongside the applied tunneling electrode voltage for reference).

FIG. 10 schematically illustrates a model one dimensional quantum mechanical double-barrier structure to be analyzed, with relevant parameters defined.

### DEFINITIONS

This invention is not limited to specific compositions, methods, steps, or equipment, as such may vary. The terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the first and second limit of that range and any other stated or intervening value in that stated range is encompassed within the invention. Also, it is contemplated that any optional feature of the inventive variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity. In the event that terms in this application are in conflict with the usage of ordinary skill in the art, the usage herein shall be controlling.

Where a range of values is provided, it is understood that each intervening value, to the tenth of the unit of the second limit unless the context clearly dictates otherwise, between the first and second limit of that range, and any other stated or intervening value in that stated range, is encompassed within the invention. The first and second limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used in this specification and the appended claims, the singular forms “a”, “an” and “the” include plural referents unless the context clearly dictates otherwise. Thus, for example, reference to “a quenchable excitable molecule” includes more than one quenchable excitable molecule, and reference to “an electrode” includes a plurality of electrodes and the like. In describing and claiming the present invention, the following terminology will be used in accordance with the definitions set out below.

A “biopolymer” is a polymer of one or more types of repeating units, regardless of the source (e.g., biological (e.g., naturally-occurring, obtained from a cell-based recombinant expression system, and the like) or synthetic). Biopolymers may be found in biological systems and particularly include polypeptides, polynucleotides, proteoglycans, edgeids, sphingoedgeids, etc., including compounds containing amino acids, nucleotides, or a mixture thereof.

The terms “polypeptide” and “protein” are used interchangeably throughout the application and mean at least two covalently attached amino acids, which includes proteins, polypeptides, oligopeptides and peptides. A polypeptide may be made up of naturally occurring amino acids and peptide bonds, synthetic peptidomimetic structures, or a mixture thereof. Thus “amino acid”, or “peptide residue”, as used herein encompasses both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the D- or the L-configuration.

In general, biopolymers, e.g., polypeptides or polynucleotides, may be of any length, e.g., greater than 2 monomers, greater than 4 monomers, greater than about 10 monomers, greater than about 20 monomers, greater than about 50 monomers, greater than about 100 monomers, greater than about 300 monomers, usually up to about 500, 1000 or 10,000 or more monomers in length. “Peptides” and “oligonucleotides” are generally greater than 2 monomers, greater than 4 monomers, greater than about 10 monomers, greater than about 20 monomers, usually up to about 10, 20, 30, 40, 50 or 100 monomers in length. In certain embodiments, peptides and oligonucleotides are between 5 and 30 amino acids in length.

The terms “polypeptide” and “protein” are used interchangeably herein. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while typically not attached in their native state, typically are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

A “monomeric residue” of a biopolymer is a subunit, i.e., monomeric unit, of a biopolymer. Nucleotides are monomeric residues of polynucleotides and amino acids are monomeric residues of polypeptides.

A “substrate” refers to any surface that may or may not be solid and which is capable of holding, embedding, attaching or which may comprise the whole or portions of an excitable molecule.

The term “nanopore” refers to a pore or hole having a minimum diameter on the order of nanometers and extending through a thin substrate. Nanopores can vary in size and can range from 1 nm to around 300 nm in diameter. Most effective nanopores have been roughly around 1.5 nm to 30 nm, e.g., 3 nm-20 nm in diameter. The thickness of the substrate through which the nanopore extends can range from 1 nm to around 700 nm.

A biopolymer that is “in”, “within” or moving through a nanopore means that the entire biopolymer any portion thereof, may located within the nanopore.

An “excitable molecule” is any molecule that may transition from ground state to singlet or triplet state and then back to ground state. An excitable molecule may comprise an aromatic or multiple conjugated double bonds with a high degree of resonance stability. These classes of substances have delocalized π electrons that can be placed in low lying excited singlet states. In addition, these molecules may also comprise quantum dots or other molecules capable of absorbing and/or releasing energy. Quantum dots also have the advantage of not photo-bleaching. The excitable molecule may comprise one or more different dyes, quantum dots or any other molecules capable of absorbing and/or releasing energy.

A “quenchable excitable molecule” is any excitable molecule that is subject to quenching, “quenching”, where “quenching” occurs when energy from an photon absorbed by a excitable molecule is transferred to a nearby energy receptor molecule rather than being re-radiated as a detectable signal (e.g., a fluorescent signal). Accordingly, when quenching occurs, an excitable molecule typically emits less signal than it would if quenching does not occur, leading to reduction in signal.

The term “resonant” or “resonant tunneling” refers to an effect where the relative energy levels between the current carriers in the electrodes are relatively similar to the energy levels of the proximal biopolymer segment. This provides for increased conductivity.

The term “ramping potential” or “bias potential” refers to having the ability to establish a variety of different voltages over time. In certain cases, this may be referred to as “scanning a voltage gradient” or providing a voltage gradient over time. A ramping potential may provided by a “ramping potential-providing element” or a “potential-providing element”.

The term “voltage gradient” refers to having the ability to establish a gradient of potentials between any two electrodes.

The term “tunneling” refers to the ability of an electron to move from a first position in space to a second position in space through a region that would be energetically excluded without quantum mechanical tunneling.

“Hybridizing”, “annealing” and “binding”, with respect to polynucleotides, are used interchangeably. “Binding efficiency” refers to the productivity of a binding reaction, measured as either the absolute or relative yield of binding product formed under a given set of conditions in a given amount of time. “Hybridization efficiency” is a particular sub-class of binding efficiency, and refers to binding efficiency in the case where the binding components are polynucleotides.

It will also be appreciated that throughout the present application, that words such as “first”, “second” are used in a relative sense only. A “set” may have one type of member or multiple different types. “Fluid” is used herein to reference a liquid. The terms “symmetric” and “symmetrized” refer to the situation in which the tunneling barriers from each electrode to the biopolymer are substantially equal in magnitude.

The terms “translocation” and “translocate” refer to movement through a nanopore from one side of the substrate to the other, the movement occurring in a defined direction.

The terms “portion” and “portion of a biopolymer” refer to a part, subunit, monomeric unit, portion of a monomeric unit, atom, portion of an atom, cluster of atoms, charge or charged unit.

In many embodiments, the methods are coded onto a computer-readable medium in the form of “programming”, where the term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

The term “adjacent” refers to anything that is near, next to or adjoining. For instance, a nanopore referred to as “adjacent to an excitable molecule” may be near an excitable molecule, it may be next to the excitable molecule, it may pass through an excitable molecule or it may be adjoining the excitable molecule. “Adjacent” can refer to spacing in linear, two-dimensional and three-dimensional space. In general, if a quenchable excitable molecule is adjacent to a nanopore, it is sufficiently close to the edge of the opening of the nanopore to be quenched by a biopolymer passing through the nanopore. Similarly, electrodes that are positions adjacent to a nanopore are positioned such that resonance tunneling occurs a biopolymer passes through the nanopore. Compositions that are adjacent may or may not be in direct contact.

If one compositions is “bound” to another composition, the bond between the compositions do not have to be in direct contact with each other. In other words, bonding may be direct or indirect, and, as such, if two compositions (e.g., a substrate and a nanostructure layer) are bound to each other, there may be at least one other composition (e.g., another layer) between to those compositions. Binding between any two compositions described herein may be covalent or non-covalent.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

### DETAILED DESCRIPTION OF THE INVENTION

The present invention provides a method and apparatus for determining the identity of a monomeric residue of a biopolymer. The apparatus comprises a substrate having a nanopore, a potential-producing element for producing a ramped potential across electrodes adjacent to the nanopore, and a quenchable excitable moiety adjacent to the nanopore. As a biopolymer passes through the nanopore, the identity of monomeric residues of a biopolymer may be determined by detecting changes in (a) current across the electrodes and (b) a signal of the quenchable excitable molecule. The subject method and apparatus find use in determining the identity of a plurality of monomeric residues of a biopolymer, and, as such, may be employed in a variety of diagnostic and research applications.

As discussed above, the invention relates to an apparatus for determining the identity of a monomeric residue of a biopolymer as the biopolymer passes through a nanopore. In general, the subject apparatus contains two signal producing components: a) a potential-producing element for producing a ramped potential across electrodes adjacent to the nanopore and b) a quenchable excitable moiety adjacent to the nanopore. As a biopolymer moves through the nanopore of a subject apparatus, signals indicating the identity of monomeric residue are produced by each of the signal producing components, and those signals may be compared to provide a highly reliable indication of the identity of a monomeric residue of the polymer. By assessing signals from several contiguous monomeric residues of the biopolymer as the biopolymer passes through the nanopore, the identity of a plurality of contiguous monomeric residues of the biopolymer may be determined. For example, the amino acid or nucleotide sequence of a biopolymer may be determined.

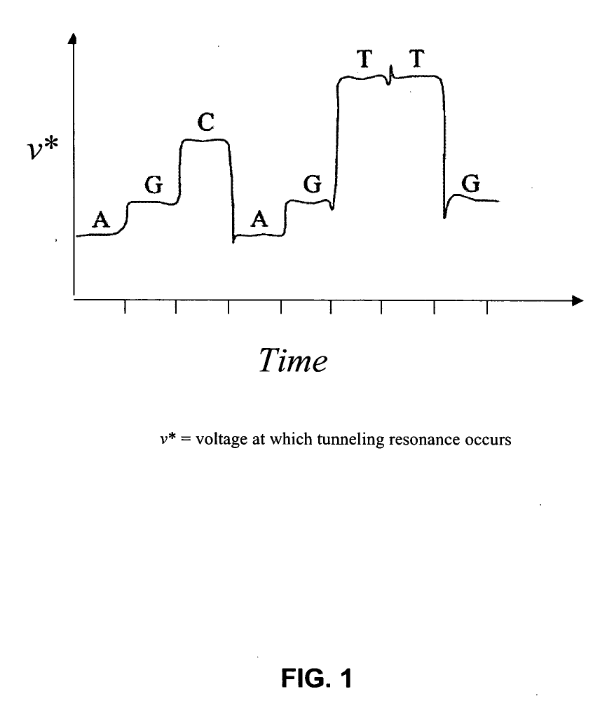

The first signal producing component of a subject apparatus identifies the monomeric residues of a biopolymer by detecting resonance tunneling. In this approach, a ramped voltage potential between two electrodes positioned adjacent to the nanopore is provided by a potential-providing element, and the current between the electrodes is detected. At specific voltages the incident energy matches the energy level of the monomeric residue positioned between the electrodes, leading to an increase in the current across the electrodes (the tunneling current). This phenomenon is termed resonance tunneling. Different monomeric residues have different energy levels, and, accordingly, the voltage at which a monomeric residue causes resonance tunneling (i.e., an increase in current) may be used to determine the identity of the monomeric residue. Accordingly, by monitoring the tunneling current as a biopolymer moves through the second signal producing component of a subject apparatus, the identity of the monomeric residues of the biopolymer can be determined. Exemplary results from this signal-producing component are shown in FIG. 1, where voltage is plotted in the x axis, and time (representing the time taken by a biopolymer to pass through the nanopore past electrodes) is plotted in they axis. Monomers G, A, T and C (e.g., nucleotides G, A, T and C) are each associated with different resonance tunneling voltages and can be discerned thereby. The sequence of the biopolymer shown in FIG. 1 is AGCAGTTG.

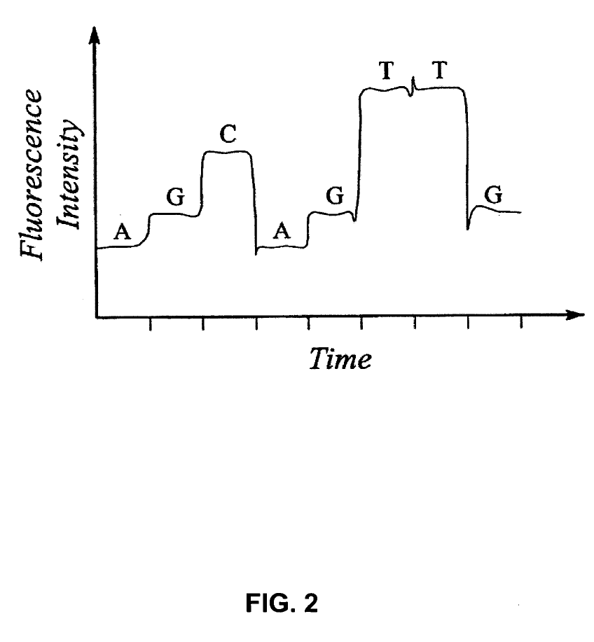

As will be discussed in greater detail below, the amplitude of a signal obtained from the quenchable excitable moiety changes as different monomeric residues of a biopolymer pass through the nanopore as a result of quenching. In other words, the monomeric residues of the biopolymer quench an excited moiety, e.g., a fluorescent or phosphorescent molecule, as the biopolymer passes through the nanopore. Since the different monomeric residues of a biopolymer have different abilities to quench an excited moiety, they can be discerned from each other by assessing the amount that the excited moiety is quenched, i.e., by assessing the reduction in excited moiety signal. Accordingly, the signal of the excited molecule changes as the different monomeric residues of a biopolymer pass by the excited molecule, and the identity of the monomeric residues of the biopolymer can be determined by measuring the excited molecule signal. Exemplary results from such a signal-producing system (i.e., the “second” signal producing system) are shown in FIG. 2, where signal intensity is plotted in the x axis, and time (representing the time taken by a biopolymer to pass through the nanopore past the quenchable excitable molecule) is plotted in they axis. Monomers G, A, T and C (e.g., nucleotides G, A, T and C) each have different signal amplitudes and can be discerned thereby. The sequence of the biopolymer shown in FIG. 2 is AGCAGTTG.

A subject apparatus therefore contains two components that each independently produce a signal that indicates the identity of a monomeric residue of a biopolymer as the biopolymer passes through a nanopore. Accordingly, the invention provides two independent indications of the identity of a monomeric residue. The two indications may be compared, e.g., by software, to provide a reliable determination of the identity of a monomeric residue of a biopolymer, and, as such, the instant apparatus represents a great improvement in the art. Further, if discrepancies between the indications are detected for one monomeric residue (e.g., where each of the two signal producing components produces a different indication), the identity of that monomeric residue may be tagged so as to indicate that that monomeric residue may be one of two monomeric residues, for example. In certain embodiments where there are discrepancies in the indications, the quality of the signals produced by the signal-producing components may be assessed, and the identity of the monomeric residue may be assigned on the basis of the highest quality signal.

Because the subject apparatus provides two independent methods for assessing the identity of a monomeric residue of a biopolymer, the apparatus produces highly reliable data and reliably predicts the identity of the biopolymeric residues as they pass through the nanopore of the apparatus.

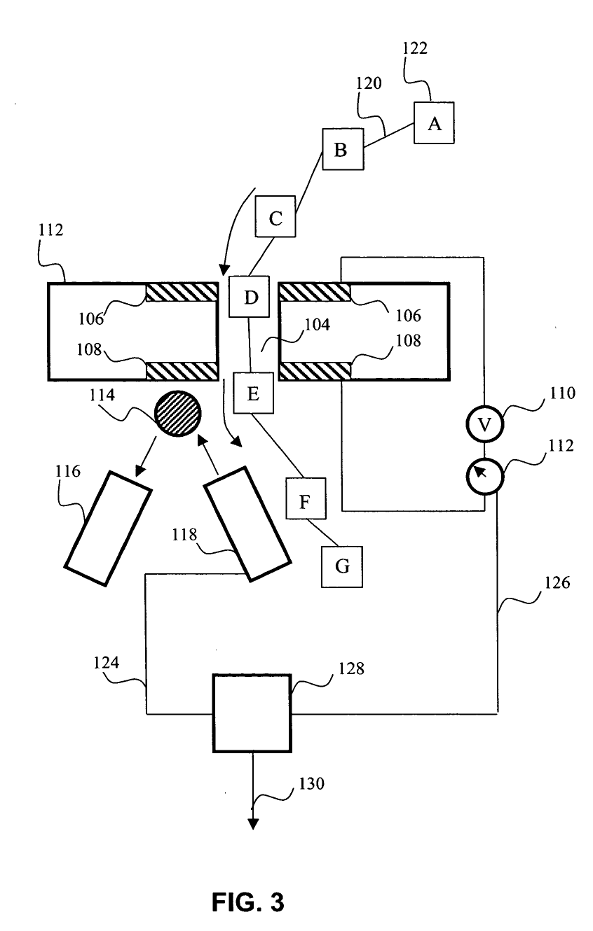

FIG. 3, showing an exemplary embodiment of the invention, illustrates several features of the invention. In viewing the embodiment shown in FIG. 3 and as explained in greater detail below, embodiments of subject apparatus other than that shown in FIG. 3 may contain different arrangements of electrodes/quenchable excitable molecules, light sources, light detectors, current detectors, and ramped potential-producing elements. Accordingly, the invention should not be limited by the embodiment shown in FIG. 3.

The apparatus shown in FIG. 3 contains a substrate 102 containing a nanopore 104. Adjacent to the nanopore are electrodes 106 and 108, which in the embodiment shown in FIG. 3 are ring electrodes that surround the openings of the nanopore. The electrodes are electrically connected to ramped voltage generator 110 and current detector 112 for detecting a resonant tunneling current, as discussed above. The particular wiring of the electrodes, ramped voltage generator and current meter may vary greatly. Also adjacent to an opening of nanopore 104 is a quenchable excitable molecule 114. Light source 116 and light detector 118 are situated so that they can excite the quenchable excitable molecule and detect a signal therefrom. Also shown in FIG. 3 is a biopolymer 120 having seven monomeric residues 122 of different identities (A-G). Biopolymer 120 is passing through nanopore 104. Biopolymer 120 may travel through nanopore 104 in any direction desired. However, in certain embodiments and as indicated by the arrow that lies next to biopolymer 120 in each of the figures, biopolymer 120 may travel through nanopore 104 such that the monomeric residues of the biopolymer are in proximity with the quenchable excitable molecule as the exit the nanopore. Electrodes 106 and 108 are sufficiently proximal to biopolymer 120 to generate a resonance tunneling current, and quenchable excitable molecule 114 is sufficiently proximal to biopolymer 120 to be quenched.

In operation, current meter 112 produces a first signal 126 indicative of the identity of the same monomeric residue of biopolymer 122 (e.g., D), and light detector 118 produces a second signal 124 indicative of the identity of a monomeric residue of a biopolymer 122 (e.g., D). The signals 124 and 126 are assessed 128 (typically by a processor), e.g., compared, to produce a single determination of the identity of the monomeric residue 130. The identity of contiguous monomer residues of the biopolymer (e.g., A-G) may be determined as biopolymer 120 passes through nanopore 194 by accumulating data for each monomer. In further describing the present invention, exemplary apparatuses of the invention will be described first, followed by a detailed description of how the apparatuses may be used to determine the identity of monomeric residues of a biopolymer. The following U.S. Patent Applications are incorporated by reference in their entirety, including all figures, detailed description and examples, for all purposes: Ser. No. 10/352,675 filed on Jan. 27, 2003 (docket no. 10030031-1) and Ser. No. 10/699,478 filed on Oct. 30, 2003 (docket no. 10020502-1).

Compositions

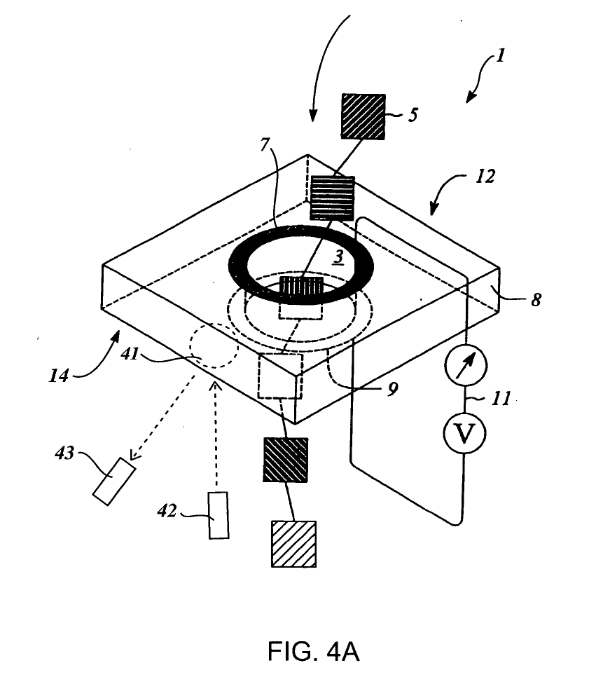

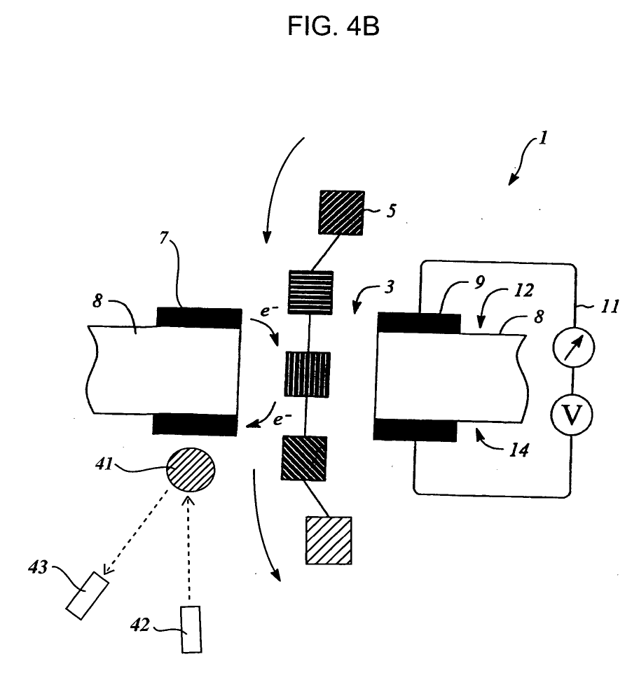

Referring now to FIGS. 4-6, the present invention provides apparatus 1 that is capable of identifying or sequencing a biopolymer 5. The biopolymer identification apparatus 1 comprises a first electrode 7, a second electrode 9, a potential means 11 and quenchable excitable molecule 41. Either or both of the electrodes may be ring shaped. The first electrode 7 and the second electrode 9 are electrically connected to the potential means 11. The second electrode 9 is adjacent to the first electrode 7 and spaced from the first electrode 7. A nanopore 3 may pass through the first electrode 7 and the second electrode 9. However, this is not a requirement of the invention. In the case that the optional substrate 8 is employed, the nanopore 3 may also pass through the substrate 8. Nanopore 3 is designed for receiving a biopolymer 5. The biopolymer 5 may or may not be translocating through the nanopore 3. When the optional substrate 8 is employed, the first electrode 7 and the second electrode 9 may be deposited on the substrate, or may comprise a portion of the substrate 8. In this embodiment of the invention, the nanopore 3 also passes through the optional substrate 8. Other embodiments of the invention may also be possible where the first electrode 7 and the second electrode 9 are positioned in the same plane (as opposed to one electrode being above or below the other) with or without the optional substrate 8. The use of multiple electrodes and/or substrates are also within the scope of the invention.

The biopolymer 5 may comprise a variety of shapes, sizes and materials. The shape or size of the molecule is not important, but it must be capable of translocation through the nanopore 3. For instance, both single stranded and double stranded RNA and DNA may be used as a biopolymer 5. In addition, the biopolymer 5 may contain groups or functional groups that are charged. Furthermore, metals or materials may be added, doped or intercalated within the biopolymer 5 to provide a net dipole, a charge or allow for conductivity through the biomolecule. The material of the biopolymer must allow for electron tunneling between electrodes. Biopolymer 5 may comprise one or more quencher moieties that quench the excitation (e.g., fluorescence) signal of the excitable molecule 41. It should be noted that the quencher moiety may comprise a portion of biopolymer 5, may be attached to biopolymer 5 or may be positioned adjacent to biopolymer 5 or attached or associated thereto. In each case the quencher moiety identifies the presence or absence of a particular base, nucleotide, peptide or monomer unit of the biopolymer 5.

Biopolymer 5 is schematically depicted as a string of beads that is threaded through nanopore 3. The biopolymer 5 typically resides in an ionic solvent such as aqueous potassium chloride, not shown, which also extends through nanopore 5. It should be appreciated that, due to Brownian motion if nothing else, biopolymer 5 is always in motion, and such motion will result in a time-varying position of each bead within the nanopore 5. The motion of biopolymer 5 will typically be biased in one direction or another through the pore by providing an external driving force, for example by establishing an electric field through the pore between a set of electrodes.

The first electrode 7 may comprise a variety of electrically conductive materials. Such materials include electrically conductive metals and alloys of tin, copper, zinc, iron, magnesium, cobalt, nickel, and vanadium. Other materials well known in the art that provide for electrical conduction may also be employed. When the first electrode 7 is deposited on or comprises a portion of the solid substrate 8, it may be positioned in any location relative to the second electrode 9. It must be positioned in such a manner that a potential can be established between the first electrode 7 and the second electrode 9. In addition, the biopolymer 5 must be positioned sufficiently close so that a portion of it may be identified or sequenced. In other words, the first electrode 7, the second electrode 9, and the nanopore 3 must be spaced and positioned in such a way that the bipolymer 5 may be identified or sequenced. This should not be interpreted to mean that the embodiment shown in FIG. 1 in any way will limit the spatial orientation and positioning of each of the components of the invention. The first electrode 7 may be designed in a variety of shapes and sizes. Other electrode shapes well known in the art may be employed. In addition, parts or curved parts of rings or other shapes may be used with the invention. The electrodes may also be designed in broken format or spaced from each other. However, the design must be capable of establishing a potential across the first electrode 7, and the nanopore 3 to the second electrode 9.

The second electrode 9 may comprise the same or similar materials as described above for the first electrode 7. As discussed above, its shape, size and positioning may be altered relative to the first electrode 7 and the nanopore 3.

Optional substrate 8 may comprise one or more layers of one or more materials including, but not limited to, membranes, edgeids, silicon nitride, silicon dioxide, platinum or other metals, silicon oxynitride, silicon rich nitride, organic polymers, and other insulating layers, carbon based materials, plastics, metals, or other materials known in the art for etching or fabricating semiconductor or electrically conducting materials. Substrate 8 need not be of uniform thickness. Substrate 8 may or may not be a solid material, and for example, may comprise in part or in whole a edged bilayer, a mesh, wire, or other material in which a nanopore may be constructed. Substrate 8 may comprise various shapes and sizes. However, it must be large enough and of sufficient width to be capable of forming the nanopore 3 through it.

The nanopore 3 may be positioned anywhere on/through the optional substrate 8. As described above, the nanopore 3 may also be established by the spacing between the first electrode 7 and the second electrode 9 (in a planar or non planar arrangement). When the substrate 8 is employed, it should be positioned adjacent to the first electrode 7 and the second electrode 9. The nanopore may range in size from 1 nm to as large as 300 nms. In most cases, effective nanopores for identifying and sequencing biopolymers would be in the range of around 2-20 nm. These size nanopores are just large enough to allow for translocation of a biopolymer. The nanopore 3 may be established using any methods well known in the art. For instance, the nanopore 3, may be sculpted in the substrate 8, using argon ion beam sputtering, etching, photolithography, or other methods and techniques well known in the art.

The potential means 11 may be positioned anywhere relative to the substrate 8, the nanopore 3, the first electrode 7 and the second electrode 9. The potential means 11 should be capable of ramping to establish a voltage gradient between the first electrode 7 and the second electrode 9. A variety of potential means 11 may be employed with the present invention. A number of these potential means are known in the art. The potential means 11 has the ability to ramp to establish a voltage gradient between the first electrode 7 and the second electrode 9. This is an important aspect of the present invention and for this reason is discussed in more detail below.

An optional means for signal detection may be employed to detect the signal produced from the biopolymer and potential means 11. This means for signal detection may be any structure, component or apparatus that is well known in the art and that may be electrically connected to one or more components of the present invention.

As noted above, the instant apparatus 1 further comprises an quenchable excitable molecule 41 which may be positioned adjacent to the nanopore 3. The biopolymer 5 may comprise one or more quencher moieties that quench a first excitation signal produced by the excitable molecule 14 after it has been irradiated by a light source 42. Modulations of the second excitation signal are detected by a detector 43 as the biopolymer 5 is translocated through the nanopore 5 in the substrate 8. Modulations of the second excitation signal are produced by the presence of one or more quencher molecules present on the biopolymer 5.

A monomeric residue of biopolymer 5 may be located near the mid-plane between quenchable excitable molecule 41 and a second quenchable excitable molecule located on the opposite side of the opening of the nanopore (not shown). If it is not in such a favorable position at one instant, the combination of Brownian motion and biased motion will ensure that it has been in such a favorable position immediately beforehand, or that it will be in such a favorable position immediately afterward. In addition, at the instant when a monomeric residue of biopolymer 5 is in the desired favorable position, the two beads adjacent thereto will not be in the desired favorable position. The use of additional excitable molecules associated with nanopore 3 is within the scope of the invention.

Quenchable excitable molecule 41 is positioned on one side of the nanopore 3, however, as noted above, other quenchable excitable molecules may also be present, e.g., on the opposite side of the nanopore opening. In general, the quenchable excitable molecule may be positioned at either end of the nanopore (i.e., either of the biopolymer entrance or exit ends). In the figures, the quenchable excitable molecule is positions at the biopolymer exit end of the nanopore (i.e., the end of the nanopore to which monomers within the nanopore travel towards as they are moving through the nanopore, as indicated by the arrow adjacent to the biopolymer). The positioning of these molecules may therefore be near the entrance of the nanopore 3 as opposed to the exit as shown in the figures. In fact, the quenchable excitable molecule may be positioned anywhere adjacent to the nanopore 3. It is important to the invention that the quenchable excitable molecule be placed in close proximity or near to the nanopore 3 so that the excitation signal (e.g. fluorescence) may be affected or modulated by the approach or presence of one a quenching monomeric subunit of a biopolymer. In this embodiment a light source 42 is employed in conjunction with the detector 43. The light source 42 irradiates the excitable molecule 41. Concomitantly, the biopolymer 3 is translocated through the nanopore 3 (in the diagram this is from the bottom to the top). The detector 43 is designed for detecting any changes in overall fluorescence output. For instance, there may be constant fluorescence or phosphorescence from the continual or pulsed irradiation of the excitable molecule 42. However, when a quencher moiety (i.e., a monomeric residue of biopolymer 5) is moved into the appropriate position in the nanopore 3, the overall signal to the detector 43 is lessened or eliminated. These fluctuations in fluorescence are determined by the detector 43. There are any number of ways of detecting such fluctuations. For instance, additional hardware, software or a combination of both may be employed with the detector 43. A background level or maximum intensity can be calibrated during the full irradiation of the excitable molecule 41. Comparisons can then be made by taking snap shots or micro spectra over time. Fluctuations can then be stored and compared. FIG. 2 shows a theoretical stochastic sensing pattern that may be obtained using such a technique. An important characteristic of the invention is for the detector to detect changes in overall fluorescence or modulation of the excitable molecules that are being irradiated by the light source 43. The various effects by these quenchers on the excitable molecules determine the overall line shape or intensity level recorded in the final spectrum.

Although the invention shows the dual application of quenchable excitable molecule 41, it is within the scope of the invention that multiple quencher molecule(s) and/or excitable molecules may be employed. The excitable molecules may be placed anywhere adjacent to the nanopore 3 and may also be placed on opposing sides of the nanopore 3. In addition, the light source 42 may be used to irradiate the excitable molecules in a sequential manner or concomitantly. Also, it is within the scope of the invention the multiple light sources may be employed on both the entrance of the nanopore 3 and/or the exit of the nanopore 3.

Quenchable excitable molecules of particular interest include fluorescent molecules that include a fluorophore moiety. Specific fluorescent molecules of interest include: xanthene dyes, e.g. fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc. NBD, fluorescein and BODIPY dyes, includig BCECF, carboxy SNARF-1, BODIPY FL and Alexa Fluor 488 dye are of particular interest.

In certain embodiments, a laser or light pipe may be employed to illuminate and excite the quenchable excitable molecule. The illumination may be pulsed or continual. In certain embodiments, a pore forming agent such as a-hemolysin may be employed to define nanopore 3. In each case, the pore must be large enough for the biopolymer 5 to translocate across substrate 8 and allow for the sequencing and detection of the base units or monomeric residues of the biopolymer.

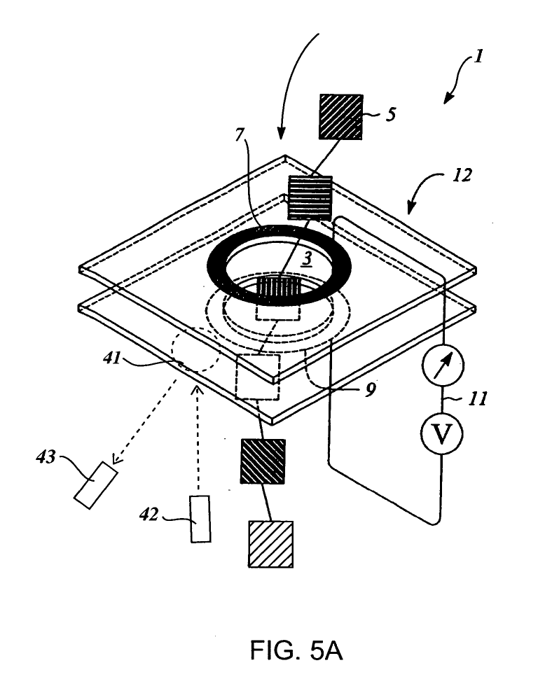

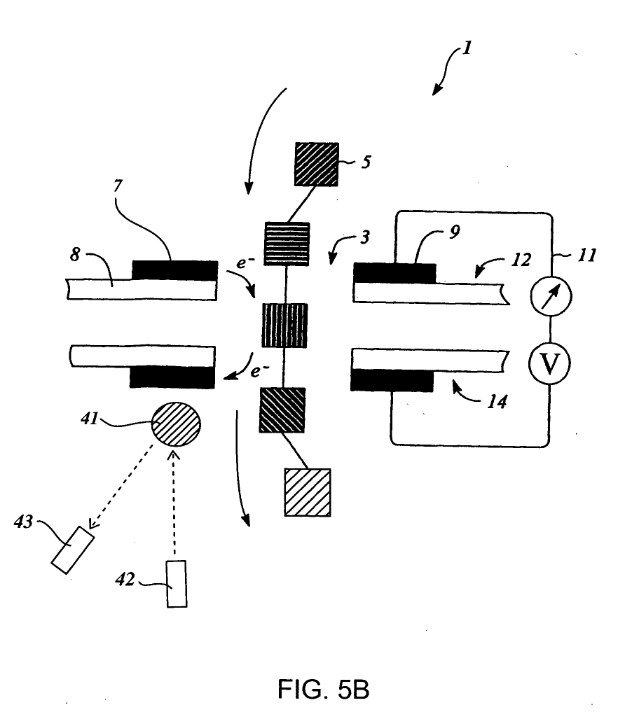

Referring now to FIGS. 5A and 5B, a second embodiment of the invention, a series of separate substrates may be employed. For instance, a first substrate 16 and a second substrate 18 may be employed in place of the single substrate 8. In this embodiment of the invention, the first electrode 7 comprises first substrate 16 or a portion of this substrate. The electrode may be embedded, attached, layered, deposited, etched on the substrate or it may comprise all or a portion of the first substrate 16. Second electrode 9 comprises the second substrate 18 or a portion of the substrate. The electrode may be embedded, attached, layered, deposited, etched on the substrate or it may comprise all or a portion of the second substrate 18. The first substrate 16 is positioned adjacent to the second substrate 18. The figure shows the first substrate 16 positioned spatially above the second substrate 18. The first electrode 7 may comprise a first nanopore 3 while the second electrode 9 may comprise a second nanopore 3′. The first nanopore 3 of the first electrode 7 and the second nanopore 3′ of the second electrode 9 may have center points that are coaxially aligned to form a single contiguous pore that the biopolymer 5 may translocate through. It is within the scope of the invention that the nanopore 3 and the nanopore 3′ center points may be offset or spaced at relative angles and distances from each other.

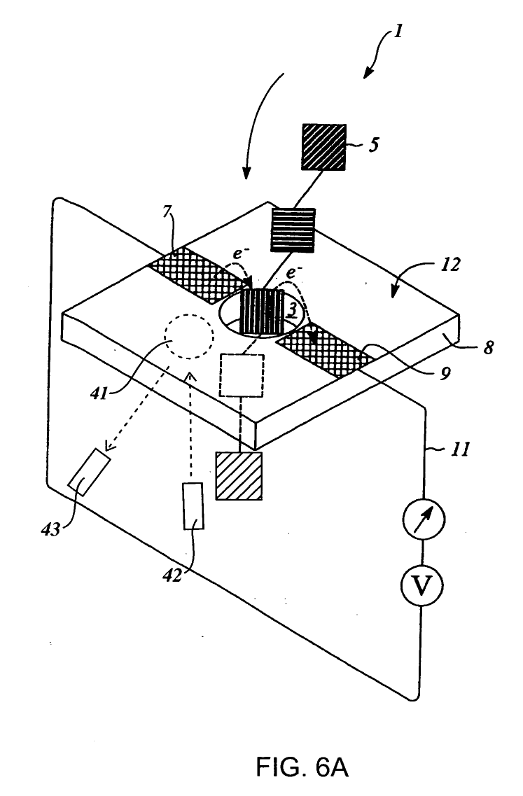

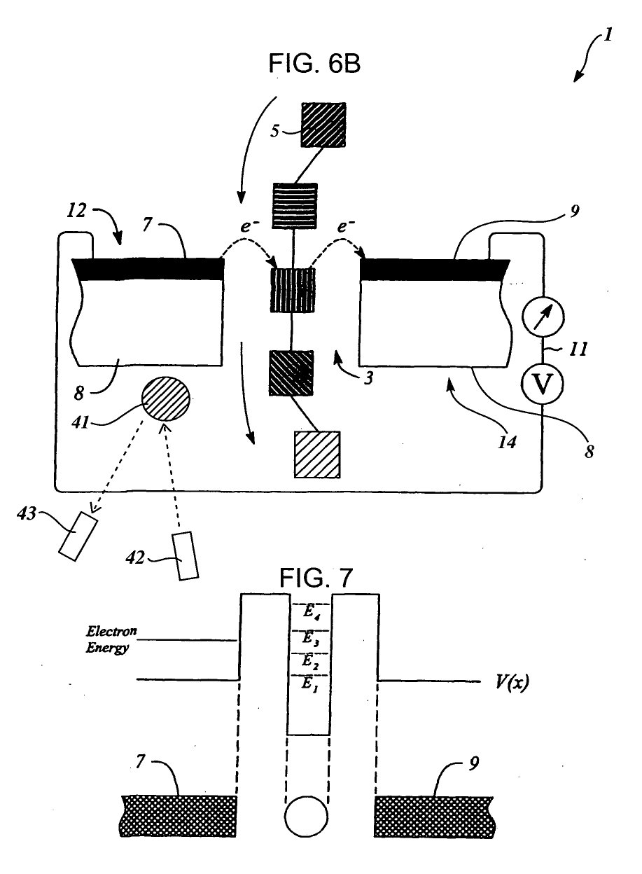

Referring now to FIGS. 6A and 6B, a third embodiment of the present invention is provided. In this embodiment, the first electrode 7 and the second electrode 9 are spaced in the same plane. One or more optional substrates or electrodes may be employed. When the optional substrate 8 is not employed, the first electrode 7 and the second electrode 9 may be positioned adjacent to define the nanopore 3. Although the figures show a pair of electrodes, the invention should not be interpreted to be limited to only this configuration. Various electrodes of varying shapes or sizes may be employed. Furthermore, it is anticipated that the invention comprises a number of similar or different electrodes capable of tunneling in a variety of directions and space (i.e. one, two and three dimensional space).

Accordingly, the subject apparatus contains two signal producing components that may each independently indicate the identity of a residue of a polymer. In certain embodiments of the invention, a subject apparatus may contain a processor (i.e., a computer processor) for comparing the results obtained from the two signal producing systems.

Having described the important components of the invention, a description of the voltage gradient and scanning of the electronic energy levels is in order. An important component of the invention is the potential means 11. As described above, the potential means 11 may be ramped. The purpose of the ramping and how it is accomplished will now be discussed.

While it is possible to imagine some differences in the tunneling current due to the size and general characteristics of a translocating monomer in the region between two conducting electrodes as illustrated in FIG. 4-6, it would be naively expected that the tunneling currents for each monomer would have qualitatively similar magnitudes, making differentiating the various monomers problematic. This is particularly true when it is considered that the biopolymer will move about laterally as it passes through the pore, significantly changing the magnitude of the tunneling current. Instead, it is proposed that to adequately differentiate the monomers, it is necessary to identify the internal structure of each individual monomer. This would be most readily accomplished by “scanning” the electronic energy level structure of each monomer as it translocates the pore. First, the physical mechanism by which it can be accomplished is described, making clear the dynamical requirements. Then, a physical realization of a structure that satisfies these requirements will be given.

It is important to have a simple model physical system that exhibits the relevant characteristics of the real system, yet is tractable. FIG. 7 shows a model of a tunneling configuration. It is a one dimensional quantum mechanical representation of the physical system, where the potential energy levels are chosen to represent the identified physical regions as shown. While the detailed shapes of the barriers and quantum well corresponding to the monomer are not important, the general characteristic of a quantum well with a distinct energy level spectrum that is separated by energy barriers from the conduction electrodes is important.

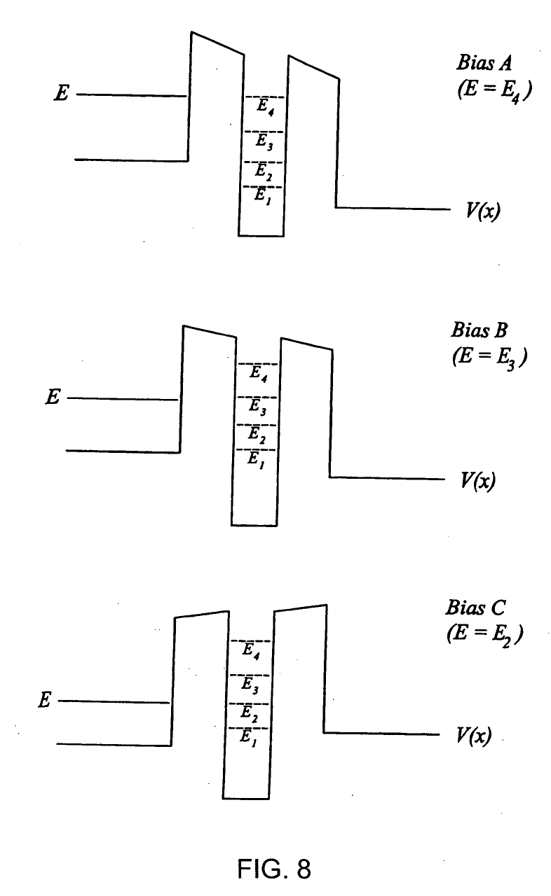

It is known from quantum mechanical calculations of Example 2, that for the double barrier potential shown in FIG. 7, the transmission probability of a particle incident upon this structure is 100% if the incident energy matches one of the bound state energies of the central quantum well. This phenomenon is called resonant tunneling, and is a central feature of the present invention. The general idea employed in the present invention is to ramp the tunneling voltage across the electrodes over the energy spectrum of the translocating biopolymer 5. As shown in FIG. 8, at specific voltages the incident energy will sequentially match the internal nucleotide energy levels, giving rise to enormous increases in the tunneling current. It is, of course, necessary that the ramp-time of the applied voltage is short compared to the nucleotide translocation time through the nanopore. Under current experimental conditions, the monomers translocate the nanopore in roughly a microsecond (See Kasianowicz et al., “Characterization of individual polynucleotide molecules using a membrane channel”, Proc. Natl. Acad. Sci. USA, 93: 13770-13773, 1996; Akeson et al., “Microsecond time-scale discrimination among polycytidylic acid, polyadenylic acid, and polyuridylic acid as homopolymers or as segments within single RNA molecules”, Biophys. J. 77: 3227-3233 (1999)). Thus, the constraint placed upon the applied tunneling voltage frequency is that it be something in excess of about 10 MHz.

A detailed study of the one-dimensional quantum mechanical double-barrier transmission problem reveals a difficulties with prior art devices. The calculations set forth in Example 2, demonstrate that the transmission probability only becomes 100% when the incident energy matches an internal energy level and the two barriers are of equal strength. This “equal barrier condition” is documented in the literature, but rarely mentioned in discussions of resonant tunneling phenomena.

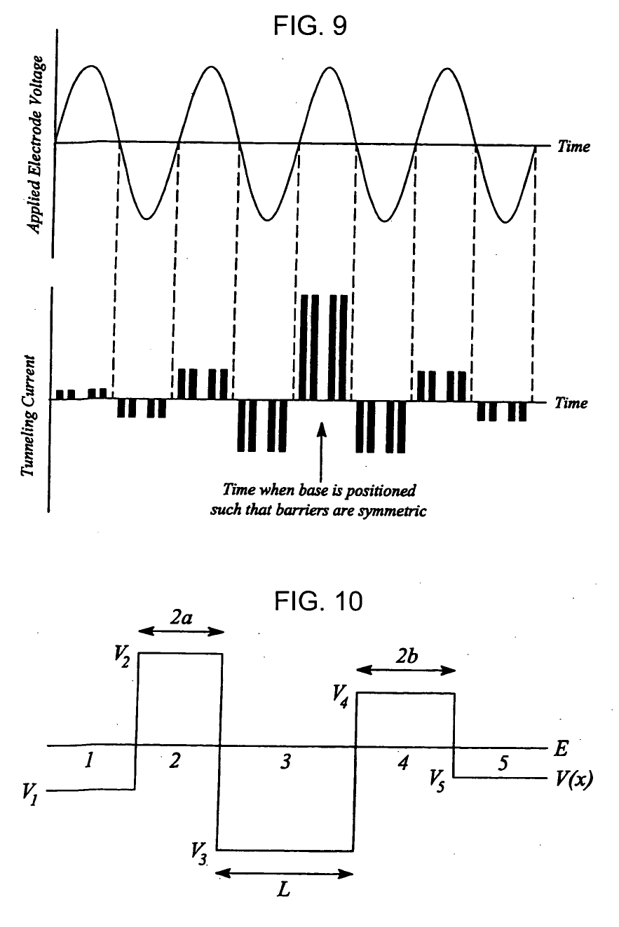

Problems with the prior art are solved by the apparatuses schematically shown in FIGS. 3-6. These apparatuses take advantage of the fact that the biopolymer 5 is in motion through the nanopore. As a monomer translocates through the nanopore and between the two ring electrodes, it will always pass a point where the barriers separating it from the two ring electrodes are equal, regardless of the origin of the initial barrier asymmetry (either spatial separation or steric asymmetry). At this point, there will be large resonant tunneling current increases as the tunneling voltage scans the internal energy spectrum of the individual monomer. A representative plot of an expected resonant tunneling current output spectrum is shown in FIG. 9 as a function of time, alongside the applied tunneling electrode voltage, for reference. As previously discussed, each type of monomer would have a characteristic internal energy level spectrum which would allow it to be distinguished from the other monomer types.

The embodiments of the ring electrode structure shown in FIGS. 3-6 are merely illustrative, and not intended to limit the scope of the present invention. For ease of fabrication, any fraction of the upper and lower surfaces could in fact be metallized, as long as the entire region surrounding the opening of the nanopore is metallized. This would obviate the need for precise alignment and placement of lithographically defined metal electrode structures.

Referring now to FIG. 10, the applied voltage and tunneling current can be seen to produce a defined signal that is indicative of the portion of the biopolymer that is proximal to the first electrode 7, or the second electrode 9. Each residue of biopolymer 5 should produce a differing signal in the tunneling current over time as the varying voltage is applied. For instance, when the monomer or portion of biopolymer 5 is positioned such that the barriers are symmetric, a larger overall signal can be seen from the tunneling current. These differing signals provide a spectrum of the portion of the biopolymer 5 that is positioned proximal to the first electrode 7, or the second electrode 9. These spectra can then be compared by computer to previous spectra or “finger prints” of nucleotides or portions of biopolymer 5 that have already been recorded. The residue of biopolymer 5 can then be determined by comparison to this database. This data and information can then be stored and supplied as output data of a final sequence.

Methods

The invention also provides methods for determining the identity of a monomeric residue of a biopolymer. In general, the methods involves moving a biopolymer such that a monomeric residue of the biopolymer is positioned in a nanopore of an apparatus comprising: (a) a substrate comprising a nanopore; (b) a potential-producing element for producing a ramped potential across electrodes adjacent to said nanopore; (c) a first detector for detecting changes in current across said electrodes as said biopolymer moves through said nanopore; (d) a quenchable excitable molecule adjacent to the nanopore; and (e) a second detector for detecting changes in a signal of the quenchable excitable molecule as said biopolymer moves through said nanopore. The method involves detecting changes in (a) the current across the electrodes and (b) the signal of the quenchable molecule to determine the identity of the monomeric residue. The above-recited elements may occur in any order, however, in certain embodiments, the residues of a biopolymer are first assessed by resonance tunneling as they enter or pass through the nanopore, and then assessed by quenching as the nanopore exits the nanopore.

In many embodiments, the method comprises a) producing a ramped potential across said electrodes; b) exciting the quenchable excitable molecule to produce a signal indicative of said monomer to provide a current indicative of said monomer; and
c) assessing (e.g., comparing) the signal and the current to provide the identity of said monomer.

By sequentially performing the above discussed methods on the contiguous monomeric residue of a biopolymer passing through a nanopore of a subject device, the identities of those residues become known.

Results obtained from the above methods may be raw results (such as signal lines for each of the signal producing systems) or may be processed results (such as those obtained by subtracting a background measurement, or an indication of the identity of a particular residue of a biopolymer (e.g., an indication of a particular nucleotide or amino acid).

In certain embodiments, the subject methods include a step of transmitting data or results from at least one of the detecting and deriving steps, also referred to herein as evaluating, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. Results obtained from the two signal producing systems of a subject apparatus may be transmitted and then compared, or the results may be compared before transmittal.

Computer-Related Embodiments

The invention also provides a variety of computer-related embodiments. Specifically, the apparatus described above may include a computer and the final “comparison” steps of the methods described in the previous section may be performed with the aid of a computer. In particular embodiments, the first and second signals indicating the identity of a particular residue of a biopolymer produced by a subject apparatus may be assessed by software (typically executed by a computer processor) to provide a final indication of the identity of that residue. If the first and second signals both indicate the same residue, then the final indication typically also indicates that residue. If the first and second signals indicate different residues, then the software may assess the quality of the first and second signals to determine the highest quality signal, and the final indication may indicate the residue indicated by the highest quality signal. In other embodiments, if the first and second signals indicate different residues, the identity of a residue may be indicated in the alternative, e.g., as “X or Y”, wherein X and Y are different monomeric residues. A quality score may be assigned to each of the third indications on the basis of the quality of the first and second signals obtained from a subject apparatus.

### EXAMPLE 1

The device can be fabricated using various techniques and materials. The nanopore can be made in a thin (500 nM) freestanding silicon nitride (SiN3) membrane supported on a silicon frame. Using a Focused Ion Beam (FIB) machine, a single initial pore of roughly 500 nM diameter can be created in the membrane. Then, illumination of the pore region with a beam of 3 KeV Argon ions sputters material and slowly closes the hole to the desired dimension of roughly 2 nM in diameter (See Li et al., “Ion beam sculpting at nanometer length scales”, Nature, 412: 166-169, 2001). Metal electrodes are formed by evaporation or other deposition means on the opposing surfaces of the SiN₃ membrane. Wire bonding to the metal electrodes allows connection to the tunneling current bias and detection system. The bias is applied using an AC source with the modest requirement of roughly 3-5 volts at 30-50 MHz. The tunneling currents are expected to be in the nanoamp range, and can be measured using a commercially available patch-clamp amplifier and head-stage (Axopatch 200B and CV203BU, Axon Instruments, Foster City, Calif.). One or more of many fluorescent molecules may be attached to the substrate near the opening of the nanopore, and those fluorescent molecules may be excited and detected using well known technology.

### EXAMPLE 2

The model physical system to be analyzed is a one-dimensional quantum mechanical double-barrier structure shown in FIG. 7. The structure is analyzed by solving the time-independent Schrodinger equation for a fixed energy incident particle, and computing the transmission probability. The parameters used in the calculations are defined in FIG. 10.

A1. Double Barrier Solution

It is assumed that the particle total energy is greater than the potential energy in all regions except the barriers. Under this condition, the solutions to the Schrodinger equation in each of the five regions defined in FIG. 10 can be written down directly
Ψ₁=A₁e^{ik}^{₁}^{x}+B₁e^{-ik}^{₁}^{x} 1(A1)
Ψ₂=A₂e^{-k}^{₂}^{x}+B₂e^{k}^{₂}^{x} 1(A2)
Ψ₃=A₃e^{ik}^{₃}^{x}+B₃e^{-ik}^{₃}^{x} 1(A3)
Ψ₄=A₄e^{-k}^{₄}^{x}+B₄e^{k}^{₄}^{x} 1(A4)
Ψ₅=A₅e^{ik}^{₅}^{x}+B₅e^{-ik}^{₃}^{x} 1(A5)
where
\overline{h}k_{1,3,5}=\sqrt{2μ(E−V_{1,3,5})} 1(A6)
\overline{h}k_{2,4}=\sqrt{2μ(·V_{2,4}−E)}. 1(A7)

The solution is determined by matching Ψ and dX/dx at the interfaces of all the homogenous regions. This procedure can be performed as a pair of subproblems. Matching the boundary conditions across the first barrier allows the wavefunction coefficient in region 1 to be written in terms of the coefficients in region 3

$$\begin{matrix}( \begin{matrix}{A}_{1} \\ {B}_{1}\end{matrix} ) = ( \begin{matrix}{M}_{11} & {M}_{12} \\ {M}_{21} & {M}_{22}\end{matrix} ) ( \begin{matrix}{A}_{3} \\ {B}_{3}\end{matrix} ) \\ where & 1 ( A8 ) \\ {M}_{11} = \frac{- {( {k}_{1}^{2} + {k}_{2}^{2} )}^{1 / 2} {( {k}_{2}^{2} + {k}_{3}^{2} )}^{1 / 2}}{4 {k}_{1} {k}_{2}} {ⅇ}^{i ( {k}_{1} + {k}_{3} ) a} ( {ⅇ}^{2 {k}_{2} a + i ( {ϕ}_{2} + {ϕ}_{3} )} - {ⅇ}^{- 2 {k}_{2} a - i ( {ϕ}_{2} + {ϕ}_{3} )} ) & 1 ( A 9 ) \\ {M}_{12} = \frac{- {( {k}_{1}^{2} + {k}_{2}^{2} )}^{1 / 2} {( {k}_{2}^{2} + {k}_{3}^{2} )}^{1 / 2}}{4 {k}_{1} {k}_{2}} {ⅇ}^{i ( {k}_{1} - {k}_{3} ) a} ( - {ⅇ}^{2 {k}_{2} a + i ( {ϕ}_{2} - {ϕ}_{3} )} + {ⅇ}^{- 2 {k}_{2} a - i ( {ϕ}_{2} - {ϕ}_{3} )} ) & 1 ( A 10 )\end{matrix}$$

M₂₂=M₁₁* 1(A11)
M₂₁=M₁₂* 1(A12)
and
φ₂=a tan(k₂/k₁) 1(A13)
φ₃=a tan(k₂/k₃). 1(A14)

Similarly, matching the boundary conditions across the second barrier allows the wavefunction coefficients in region 3 to be written in terms of the coefficients in region 5

$$\begin{matrix}( \begin{matrix}{A}_{3} \\ {B}_{3}\end{matrix} ) = ( \begin{matrix}{N}_{11} & {N}_{12} \\ {N}_{21} & {N}_{22}\end{matrix} ) ( \begin{matrix}{A}_{5} \\ {B}_{5}\end{matrix} ) \\ where & 1 ( A15 ) \\ {N}_{11} = \frac{- {( {k}_{3}^{2} + {k}_{4}^{2} )}^{1 / 2} {( {k}_{4}^{2} + {k}_{3}^{2} )}^{1 / 2}}{4 {k}_{3} {k}_{4}} {ⅇ}^{- {ik}_{3} ( a + L ) + {ik}_{3} b} ( {ⅇ}^{2 {k}_{4} b + i ( {ϕ}_{4} + {ϕ}_{3} )} - {ⅇ}^{- 2 {k}_{4} b - i ( {ϕ}_{4} + {ϕ}_{3} )} ) & 1 ( A 16 ) \\ {N}_{12} = \frac{- {( {k}_{3}^{2} + {k}_{4}^{2} )}^{1 / 2} {( {k}_{4}^{2} + {k}_{3}^{2} )}^{1 / 2}}{4 {k}_{3} {k}_{4}} {ⅇ}^{- {ik}_{3} ( a + L ) - {ik}_{3} b} ( - {ⅇ}^{2 {k}_{4} b + i ( {ϕ}_{4} - {ϕ}_{3} )} + {ⅇ}^{- 2 {k}_{4} b - i ( {ϕ}_{4} - {ϕ}_{3} )} ) & 1 ( A 17 )\end{matrix}$$

N₂₂=N₁₁* 1(A18)
N₂₁=N₁₂* 1(A19)
and
φ₄=a tan(k₄/k₃) 1(A20)
φ₅=a tan(k₄/k₅). 1(A21)

The full expression connecting the wavefunction coefficients of region 1 with those of region 5 is determined by concatenating the matrices of equations (A8) and (A15).

$$( \begin{matrix}{A}_{1} \\ {B}_{1}\end{matrix} ) = ( \begin{matrix}{M}_{11} & {M}_{12} \\ {M}_{21} & {M}_{22}\end{matrix} ) ( \begin{matrix}{N}_{11} & {N}_{12} \\ {N}_{21} & {N}_{22}\end{matrix} ) ( \begin{matrix}{A}_{5} \\ {B}_{5}\end{matrix} ) . \qquad 1 ( A22 )$$

The full transmission coefficient is determined by applying the boundary condition

$$( \begin{matrix}{A}_{1} = 1 \\ {B}_{1}\end{matrix} ) = ( \begin{matrix}{M}_{11} & {M}_{12} \\ {M}_{21} & {M}_{22}\end{matrix} ) ( \begin{matrix}{N}_{11} & {N}_{12} \\ {N}_{21} & {N}_{22}\end{matrix} ) ( \begin{matrix}{A}_{5} \\ {B}_{5} = 0\end{matrix} ) \qquad 1 ( A23 )$$

which corresponds to an incident wave of unit amplitude from the left (A₁=1) and no wave incident from the right (B₅=0). Thus the calculated probability flux transmission is given by

$${T}_{tot} = \frac{{k}_{5}}{{k}_{1}} {\vert \frac{1}{{M}_{11} {N}_{11} + {M}_{12} {N}_{21}} \vert}^{2} . \qquad 1 ( A24 )$$

Performing the required algebra to explicitly evaluate equation (A24), and collecting and grouping terms which are listed in descending powers of the large “barrier suppression factors”

$${T}_{tot} = \frac{{2}^{6} {k}_{1} {k}_{2}^{2} {k}_{3}^{2} {k}_{4}^{2} {k}_{5}}{( {k}_{1}^{2} + {k}_{2}^{2} ) ( {k}_{2}^{2} + {k}_{3}^{2} ) ( {k}_{3}^{2} + {k}_{4}^{2} ) ( {k}_{4}^{2} + {k}_{5}^{2} )} \frac{1}{F} \qquad 1 ( A25 )$$

where
F=e^{2γ}^{₂}^{+2γ}^{₄} sin²(φ₁−φ₃−φ₄)+
e^{2γ}^{₂} cos(2φ₅)(−cos(2φ₄)+cos(2φ₁−2φ₃))+e^{2γ}^{₄} cos(2φ₂)(−cos(2φ₃)+cos(2φ₁−2φ₄))+e^{2γ}^{₂}^{-2γ}^{₄} sin²(φ₁−φ₃+φ₄)+e^{2γ}^{_{4−2γ}}^{₂} sin²(φ₁+φ₃−φ₄)+e⁰ cos(2φ₂−2φ₅)(−cos(2φ₁)+cos(2φ₃−2φ₄))+e⁰ cos(2φ₂+2φ₅)(−cos(2φ₁)+cos(2φ₃+2φ₄))+e^{-2γ}^{₄} cos(2φ₂)(−cos(2φ₃)+cos(2φ₁+2φ₄))+e^{-2γ}^{₂} cos(2φ₅)(−cos(2φ₄)+cos(2φ₁+2φ₃))++e^{-2γ}^{₂}^{-2γ}^{₄} sin²(φ₁+φ₃+φ₄) 1(A26)
and the following definitions have been used for relational simplicity
   φ₁≡k₃L 1(A27)
   γ₂≡2k₂a 1(A28)
   γ₄≡2k₄b. 1(A29)
   A2. Resonance Condition

Assuming the barriers are strong impediments to particle transmission, i.e. e^{2γ2}, e^{2γ4}>>1, for general “non-resonant” conditions the total transmission is dominated by the first term in equation (A26), yielding
Tₜₒₜ−e^{-2γ}^{₂}^{-2γ}^{₄}−T_{L}R_{T}. 1(A30)

For this case, the total transmission is proportional to the product of the transmissions of the two barriers separately. However, for the particular situation that
φ₁−φ₃−φ₄=nπ, 1(A31)
the coefficients of the first three terms in equation (A26) vanish. If the two barriers are of equal integrated magnitudes, i.e., γ2=γ4, then the leading term in equation (A26) is of order e°˜1, and the total transmission coefficient can be shown to approach 1. This is the condition called resonant tunneling, and exhibits the remarkable property of total transmission through a double-barrier structure, regardless of the strengths of the individual barriers (as long as they are equal).

It is important to understand the physical significance of the so-called resonance condition stated in equation (A31). For ease of analyzing this condition, we will restrict our attention to the completely symmetric case
φ₃=a tan(k₂/k₃)=a tan(k₄/k₃)=4 1(A32)
leading to
sin(φ₁−2φ₃)=0. 1(A33)

Applying simple trigonometric identities, and inserting the definitions of Φ1 and Φ3, equation (A33) can be rewritten as

$$tan ( {k}_{3} L ) = \frac{\sqrt{{V}_{2} - E} \sqrt{E - {V}_{3}}}{E - ( {V}_{2} + {V}_{3} ) / 2} . \qquad 1 ( A 34 )$$

If the arbitrary baseline potential energy level is chosen as V₃≡0 and V₂ is renamed V₀, equation (A34) take the form

$$tan ( {k}_{3} L ) = \frac{\sqrt{{V}_{0} - E} \sqrt{E}}{E - {V}_{0} / 2} . \qquad 1 ( A35 )$$

It is recognized that this condition is precisely the eigenvalue equation for the energy levels of a square well potential with the parameters stated above (See Landau and Lifshitz, “Quantum Mechanics”, Pergamon, Oxford (1989)). This demonstrates why this phenomenon of total transmission through the double-well structure is called resonant tunneling. The condition of resonant tunneling is precisely that the energy of the incident particle must match the resonant energy of the central potential well. Whenever the incident energy matches any of the resonant energies, the total particle transmission increases dramatically, as long as the double-barriers are symmetric.

A3. Tunneling Current on Resonance

As described above, for a symmetric potential structure, the transmission probability becomes unity when the incident particle energy passes through a resonance of the central well. However, the situation is markedly different for a double-barrier structure that has asymmetric barriers. For the general asymmetric structure on resonance, it is seen from equation (A26) that the leading behavior has the form
F−e^{2γ}^{₂}^{-2γ}^{₄} sin²(φ₁−φ₃+φ₄)+e^{2γ}^{₄}^{-2γ}^{₃} sin²(φ₁+φ₃−φ₄). 1(A36)

This implies that for the situation where the left barrier is larger (γ2>>γ4)

$${T}_{tot} - {ⅇ}^{2 {γ}_{4} - 2 {γ}_{2}} - \frac{{T}_{L}}{{T}_{R}} \qquad 1 ( A37 )$$

and for the situation where the right barrier is larger (γ4>>γ2)

$${T}_{tot} - {ⅇ}^{2 {γ}_{2} - 2 {γ}_{4}} - \frac{{T}_{R}}{{T}_{L}} . \qquad 1 ( A38 )$$

This demonstrates the markedly different resonant tunneling behavior for the asymmetric double-barrier structure. If the barrier is highly asymmetric, there is very little gain in the tunneling probability as the resonance condition is approached. It is only under the condition of double-barrier symmetry that the resonant tunneling phenomenon of barrier transparency is in effect.

I claim:

1. A biopolymer detection device comprising:
(a) a substrate comprising a nanopore;
(b) a potential-producing element for producing a ramped potential across electrodes adjacent to said nanopore; and
(c) a quenchable excitable moiety adjacent to said nanopore.

2. The biopolymer detection device of claim 1, further comprising:
(d) a first detector for detecting changes in current across said electrodes as a biopolymer moves through said nanopore; and
(e) a second detector for detecting changes in a signal of said excitable moiety as a biopolymer moves through said nanopore.

3. The biopolymer detection device of claim 1, wherein said nanopore has a biopolymer entrance end and a biopolymer exit end and wherein said quenchable excitable moiety is adjacent to said exit end of said nanopore.

4. The biopolymer detection device of claim 1, wherein said potential producing element comprises a first electrode, a second electrode and means for applying a ramping potential from said first electrode, via a portion of a biopolymer present in said nanopore, to said second electrode to produce a signal indicative of said portion of said biopolymer.

5. The biopolymer detection device of claim 4, wherein said first and second electrodes are ring electrodes.

6. The biopolymer detection device of claim 5, wherein said first and second ring electrodes lie at either end of said nanopore and define the openings of said nanopore.

7. The biopolymer detection device of claim 4, wherein said first and second electrodes are planar with an opening of said nanopore, and are positioned on either said of said opening.

8. The biopolymer detection device of claim 1, wherein said quenchable excitable moiety is a fluorescent, phosphorescent or luminescent moiety.

9. The biopolymer detection device of claim 2, wherein said biopolymer is a polypeptide, a polynucleotide or a polysaccharide.

10. The biopolymer detection device of claim 1, wherein said nanopore is from about 1 nanometer to about 10 nanometers in diameter.

11. The biopolymer detection device of claim 2, wherein said biopolymer comprises a quenching moiety.

12. The biopolymer detection device of claim 1, further comprising a light source for exciting said quenchable excitable moiety.

13. The biopolymer detection device of claim 12, wherein said light source is a laser.

14. The biopolymer detection device of claim 12, wherein said light source is a light pipe.

15. An biopolymer detection device comprising:
(a) a substrate comprising a nanopore;
(b) a potential-producing element for producing a ramped potential across electrodes adjacent to said nanopore;
(c) a first detector for detecting changes in current across said electrodes as said biopolymer moves through said nanopore;
(d) a quenchable excitable molecule adjacent to said nanopore;
(e) a second detector for detecting changes in a signal of said excitable molecule as said biopolymer moves through said nanopore;
(f) a light source; and
(g) a computer processor.

16. A method for determining the identity of a monomeric residue of a biopolymer, comprising:
i) moving a biopolymer such that a monomeric residue of said biopolymer is positioned in a nanopore of an biopolymer detection device comprising:
(a) a substrate comprising said nanopore;
(b) a potential-producing element for producing a ramped potential across electrodes adjacent to said nanopore;
(c) a first detector for detecting changes in current across said electrodes as said biopolymer moves through said nanopore;
(d) a quenchable excitable molecule adjacent to said nanopore; and
(e) a second detector for detecting changes in a signal of said quenchable excitable molecule as said biopolymer moves through said nanopore; and
ii) detecting changes in said current and said signal of said quenchable molecule across said electrodes to determine the identity of said monomeric residue.

17. The method of claim 16, wherein said step (ii) comprises:
a) producing a ramped potential across said electrodes to provide a current indicative of said monomer;
b) exciting said quenchable excitable molecule to produce a signal indicative of said monomer; and
c) assessing said signal and said current to provide the identity of said monomer.

18. The method of claim 17, wherein said assessing comprises comparing the identity of a monomeric residue indicated by said current to the identity of a monomeric residue indicated by said signal.

19. A method for determining the identities of a plurality of contiguous monomeric residues of a biopolymer, comprising:
i) moving said biopolymer through a nanopore of an biopolymer detection device comprising:
(a) a substrate comprising said nanopore;
(b) a potential-producing element for producing a ramped potential across electrodes adjacent to said nanopore;
(c) a first detector for detecting changes in current across said electrodes as said biopolymer moves through said nanopore;
(d) a quenchable excitable molecule adjacent to said nanopore; and
(e) a second detector for detecting changes in a signal of said quenchable excitable molecule as said biopolymer moves through said nanopore; and
ii) detecting changes in (a) current across said electrodes and (b) signal of said quenchable molecule to determine the identity of said plurality of contiguous monomeric residues.

20. The method of claim 19, wherein said method determines at least part of the monomeric residue sequence of said biopolymer.

21. The method of claim 20, wherein said biopolymer is a nucleic acid or a polypeptide.

22. A computer readable medium comprising programming to compare changes in (a) signal of a quenchable excitable molecule and (b) current across electrodes, to determine the identity of a plurality of contiguous monomeric residues.
